(19) United States
(12) Patent Application Publication
(10) Pub. No.: US 2016/0319246 A1
(43) Pub. Date: Nov. 3, 2016

(54) FLAVIN-BINDING GLUCOSE DEHYDROGENASE EXHIBITING IMPROVED HEAT STABILITY

(71) Applicant: KIKKOMAN CORPORATION, Noda-shi, Chiba (JP)

(72) Inventor: Yasuko ARAKI, Noda-shi (JP)

(73) Assignee: KIKKOMAN CORPORATION, Noda-shi, Chiba (JP)

(21) Appl. No.: 15/108,528

(22) PCT Filed: Dec. 26, 2014
(86) PCT No.: PCT/JP2014/084480

§ 371 Date: Jun. 27, 2016

(30) Foreign Application Priority Data
Dec. 27, 2013 (JP) 2013-270871

(51) Int. Cl. C12N 9/04; C12Q 1/54; C12Q 1/32

(52) U.S. Cl. CPC C12N 9/0006; C12Q 1/32; C12Y 101/9901; C12Q 1/54

(57) ABSTRACT

The present invention provides a flavin-binding glucose dehydrogenase that exhibits heat stability and has one or more amino acid substitutions at positions corresponding to positions 66, 68, 88, 158, 233, 385, 391 and 557 in the amino acid sequence set forth in SEQ ID NO: 1.

### TECHNICAL FIELD

The present invention relates to a flavin-binding glucose dehydrogenase having superior heat stability, a method for measuring glucose using the same, and a method for producing a flavin-binding glucose dehydrogenase.

### BACKGROUND ART

Blood glucose concentration (blood glucose level) is an important marker for diabetes. Devices for self-monitoring of blood glucose (SMBG) employing an electrochemical biosensor are widely used by diabetes patients to monitor their own blood glucose levels. The biosensors used in SMBG devices have conventionally used an enzyme such as glucose oxidase (GOD) that uses glucose as a substrate. However, since GOD has the property of using oxygen as an electron acceptor, in SMBG devices using GOD, dissolved oxygen present in a measurement sample affects measured values, and cases can occur in which accurate measured values are unable to be obtained.

On the other hand, various types of glucose dehydrogenases (GDH) are known to be enzymes that use glucose as a substrate but do not use oxygen as an electron acceptor. Specific examples of such enzymes that have been found include GDH (NAD(P)-GDH), which is a type that uses nicotinamide adenine dinucleotide (NAD) or nicotinamide adenine dinucleotide phosphate (NADP) as a coenzyme, and GDH (PQQ-GDH), which is a type that uses pyrroloquinoline quinone (PQQ) as a coenzyme, and these enzymes are used in the biosensors of SMBG devices. However, NAD(P)GDH has the problems of lacking enzyme stability and requiring the addition of coenzyme, while PQQ-GDH has the problem of low substrate specificity, and as a result of acting on sugar compounds other than the glucose targeted for measurement such as maltose, D-galactose or D-xylose, allows sugar compounds other than glucose present in a measurement sample to have an effect on measured values, thereby preventing the obtaining of accurate measured values.

Cases have been reported in recent years in which, when blood sugar levels of diabetes patients receiving an infusion were measured using an SMBG device employing PQQ-GDH for the biosensor, the PQQ-GDH also acted on maltose contained in the infusion liquid resulting in the obtaining of measured values that were higher than the actual blood glucose levels, and causing the occurrence of hypoglycemia as a result of the patients being treated on the basis of those measured values. In addition, similar events have been determined to also have the potential to occur when performing galactose loading tests or xylose absorption tests (see, for example, Non-Patent Document 1). When the Pharmaceutical and Food Safety Bureau of the Ministry of Health, Labour and Welfare conducted cross-reactivity tests for the purpose of investigating the effects on measured blood glucose values in the case of having added various sugars to glucose solutions, measured values obtained with a blood glucose assay kit using the PQQ-GDH method demonstrated values that were 2.5 to 3 times higher than the actual glucose concentrations in the case of having added maltose at 600 mg/dL, D-galactose at 300 mg/dL or D-xylose at 200 In other words, measured values were determined to become inaccurate due to maltose, D-galactose or D-xylose, which may be present in measurement samples, thereby resulting in the urgent desire to develop a GDH that is unaffected by such sugar compounds that cause measurement error and has a high level of substrate specificity that allows specific measurement of glucose.

In view of the circumstances described above, efforts have focused on GDH of a type that uses a coenzyme other than those described above. For example, although not describing details regarding substrate specificity, reports are known regarding GDH derived from Aspergillus oryzae (see, for example, Non-Patent Documents 2 to 5). In addition, glucose dehydrogenase (FAD-GDH) derived from Aspergillus species and Penicillium species has been disclosed that uses flavin adenine dinucleotide (FAD) as coenzyme (see, for example, Patent Documents 1 to 3), and FAD-GDH derived from. Aspergillus species has been disclosed that reduces enzymatic action on D-xylose (see, for example, Patent Document 4).

However, although the aforementioned enzymes have the properties of demonstrating low reactivity with one or more sugar compounds that are not D-glucose, they do not have the property of having sufficiently low reactivity with maltose, D-galactose or D-xylose. In contrast, FAD-GDH discovered in a type of mold in the form of Mucor species was shown to have the superior property of having sufficiently low reactivity with maltose, D-galactose and D-xylose (see, for example, Patent Document 5). The use of this GDH makes it possible to accurately measure glucose concentration without being affected by other sugar compounds even under conditions in which maltose, D-galactose and D-xylose are present (see, for example, Patent Document 5). This superior substrate specificity is one of the characteristics that indicate the practical superiority of Mucor-derived FAD-GDH. Moreover, Patent Document 5 also discloses the gene sequence and amino acid sequence of Mucor-derived FAD-GDH, and the recombinant expression in an E. coli or koji mold host using the gene sequence of Mucor-derived FAD-GDH.

When considering the application of FAD-GDH to a blood glucose sensor, since there are cases in which the sensor chip production process includes a step in which the enzyme is subjected to heat drying treatment, the FAD-GDH is required to have a high level of heat resistance. With respect to this objective, Patent Document 6 describes the discovery of Mucor-derived FAD-GDH having superior substrate specificity and heat resistance (expressed in yeast of the genus Zygosaccharomyces). In addition, Patent Document 7 discloses that the heat resistance of Mucor-derived FAD-GDH is improved by introducing a site-directed mutation.

However, when anticipating the possibility of sensor chips being subjected to harsh heat conditions during the production thereof, continuing efforts to impart greater heat stability are required.

### PRIOR ART DOCUMENTS

### Paten t Documents

Patent Document 1: Japanese Unexamined Patent Publication No. 2007-289148

Patent Document 2: Japanese Patent No. 4494978

Patent Document 3: International Publication No. WO 07/139013

Patent Document 4: Japanese Unexamined Patent Publication No. 2008-237210

Patent Document 5: Japanese Patent No. 4648993

Patent Document 6: International Publication No. WO 12/073986

Patent Document 7: International Publication No. WO 12/169512

### Non-Patent Documents

Non-Patent Document 1: Pharmaceuticals and Medical Devices Safety Information No. 206, October 2004, Pharmaceutical and Food Safety Bureau, Ministry of Health, Labour and Welfare

Non-Patent Document 2: Studies on the glucose dehydrogenase of Aspergillus oryzae: I. Induction of its synthesis by p-benzoquinone and hydroquinone, T. C. Bak and R. Sato, Biochim. Biophys. Acta, 139, 265-276 (1967)

Non-Patent Document 3: Studies on the glucose dehydrogenase of Aspergillus oryzae: II. Purification and physical and chemical properties, T. C. Bak, Biochim. Biophys. Acta, 139, 277-293 (1967)

Non-Patent Document 4: Studies on the glucose dehydrogenase of Aspergillus oryzae: III. General enzymatic properties, T. C. Bak, Biochim. Biophys. Acta, 146, 317-327 (1967)

Non-Patent Document 5: Studies on the glucose dehydrogenase of Aspergillus oryzae: IV. Histidyl residue as an active site, T. C. Bak and R, Sato, Biochim, Biophys. Acta, 146, 328-335 (1967)

### DISCLOSURE OF THE INVENTION

### Problems to be Solved by the Invention

An object of the present invention is to provide an FAD-GDH having improved heat stability.

### Means for Solving the Problems

As a result of conducting extensive studies to solve the aforementioned problems by searching for an FAD-GDH having improved heat stability, the inventor of the present invention found that an FAD-GDH having improved heat stability is obtained by introducing a mutation into a known FAD-GDH.

Namely, the present invention relates to that described below, (1) An FAD-GDH comprised of the amino acid sequence represented by SEQ ID NO: 1, an amino acid sequence having a sequence identity of 70% or more with the amino acid sequence represented by SEQ ID NO: 1, or an amino acid sequence in which one or a plurality of amino acids have been deleted, substituted or added in that amino acid sequence (amino acid sequence represented by SEQ ID NO: 1 or amino acid sequence having a sequence identity of 70% or more with the amino acid sequence represented by SEQ ID NO: 1), having one or more amino acid substitutions at positions corresponding to amino acids selected from the group indicated below, and having improved heat stability in comparison with prior to carrying out that substitution:

the amino acid at position 66 in the amino acid sequence set forth in SEQ ID NO: 1,

the acid at position 68 in the amino acid sequence set forth in SEQ ID NO: 1,

the amino acid at position 88 in the amino acid sequence set forth in SEQ ID NO: 1,

the amino acid at position 158 in the amino acid sequence set forth in SEQ NO: 1,

the amino acid at position 233 in the amino acid sequence set forth in SEQ ID NO: 1,

the amino acid at position 385 in the amino acid sequence set forth in SEQ ID NO: 1,

the amino acid at position 391 in the amino acid sequence set forth in SEQ ID NO: 1, and

the amino acid at position 557 in the amino acid sequence set forth in SEQ ID NO: 1.

(2) An FAD-GDH comprised of the amino acid sequence represented by SEQ ID NO: 1, an amino acid sequence having a sequence identity of 70% or more with the amino acid sequence represented by SEQ ID NO: 1, or an amino acid sequence in which one or a plurality of amino acids have been deleted, substituted or added in the amino acid sequence represented by SEQ ID NO: 1 or an amino acid sequence having a sequence identity of 70% or more with the amino acid sequence represented by SEQ ID NO: 1, and having one or more of the amino acid substitutions at positions corresponding to amino acids selected from the group indicated below:

the amino acid at position corresponding to position 66 in the amino acid sequence set forth in SEQ ID NO: 1 is tyrosine,

the amino acid at position corresponding to position 68 in the amino acid sequence set forth in SEQ ID NO: 1 is glycine,

the amino acid at position corresponding to position 88 in the amino acid sequence set forth in SEQ ID NO: 1 is alanine,

the amino acid at position corresponding to position 158 in the amino acid sequence set forth in SEQ ID NO: 1 is histidine,

the amino acid at position corresponding to position 233 in the amino acid sequence set forth in SEQ ID NO: 1 is arginine,

the amino acid at position corresponding to position 385 in the amino acid sequence set forth in SEQ ID NO: 1 is threonine,

the amino acid at position corresponding to position 391 in the amino acid sequence set forth in SEQ ID NO: 1 is isoleucine, and

the amino acid at position corresponding to position 557 in the amino acid sequence set forth in SEQ ID NO: 1 is valine.

(3) An FAD-GDH in the form of a modified protein wherein the amino acid at the position corresponding to the asparagine residue at position 66 in an amino acid sequence composing a protein having FAD-GDH activity indicated below is substituted with tyrosine:

a protein having FAD-GDH activity comprised of the amino acid sequence set forth in SEQ ID NO: 1, or

a protein having FAD-GDII activity comprised of amino acids in which one or a plurality of amino acids other than the amino acid residue at the position corresponding to the asparagine residue at position 66 in the amino acid sequence of SEQ ID NO: 1 has been deleted, substituted or added.

(4) An FAD-GDH in the fowl of a modified protein wherein the amino acid at the position corresponding to the asparagine residue at position 68 in an amino acid sequence composing a parent protein having FAD-GDH activity indicated below is substituted with glycine:

a protein having FAD-GDH activity comprised of the amino acid sequence set forth in SEQ ID NO: 1, or

a protein having FAD-GDH activity comprised of amino acids in which one or a plurality of amino acids other than the amino acid residue at the position corresponding to the asparagine residue at position 68 in the amino acid sequence of SEQ ID NO: 1 has been deleted, substituted or added.

(5) An FAD-GDH in the form of a modified protein wherein the amino acid at the position corresponding to the cysteine residue at position 88 in an amino acid sequence composing a parent protein having FAD-GDH activity indicated below is substituted with alanine:

a protein having FAD-GDH activity comprised of the amino acid sequence set forth in SEQ NO: 1, or

a protein having FAD-GDH activity comprised of amino acids in which one or a plurality of amino acids other than the amino acid residue at the position corresponding to the cysteine residue at position 88 in the amino acid sequence of SEQ ID NO: 1 has been deleted, substituted or added.

(6) An FAD-GDH in the form of a modified protein wherein the amino acid at the position corresponding to the threonine residue at position 158 in an amino acid sequence composing a parent protein having FAD-GDH activity indicated below is substituted with histidine:

a protein having FAD-GDH activity comprised of the amino acid sequence set forth in SEQ ID NO: 1, or

a protein having FAD-GDH activity comprised of amino acids in which one or a plurality of amino acids other than the amino acid residue at the position corresponding to the threonine residue at position 158 in the amino acid sequence of SEQ ID NO: 1 has been deleted, substituted or added.

(7) An FAD-GDH in the form of a modified protein wherein the amino acid at the position corresponding to the glutamine residue at position 233 in an amino acid sequence composing a protein having FAD-GDH activity indicated below is substituted with arginine:

a protein having FAD-GDH activity comprised of the amino acid sequence set forth in SEQ ID NO: 1, or

a protein having FAD-GDH activity comprised of amino acids in which one or a plurality of amino acids other than the amino acid residue at the position corresponding to the glutamine residue at position 233 in the amino acid sequence of SEQ ID NO: 1 has been deleted, substituted or added.

(8) A flavin-binding glucose dehydrogenase in the form of a modified protein wherein the amino acid at the position corresponding to the alanine residue at position 385 in an amino acid sequence composing a protein having flavin-binding glucose dehydrogenase activity indicated below is substituted with threonine:

a protein having flavin-binding glucose dehydrogenase activity comprised of the amino acid sequence set forth in SEQ ID NO: 1, or

a protein having flavin-binding glucose dehydrogenase activity comprised of amino acids in which one or a plurality of amino acids other than the amino acid residue at the position corresponding to the alanine residue at position 385 in the amino acid sequence of SEQ ID NO: 1 has been deleted, substituted or added.

(9) A flavin-binding glucose dehydrogenase in the form of a modified protein wherein the amino acid at the position corresponding to the leucine residue at position 391 in an amino acid sequence composing a protein having flavin-binding glucose dehydrogenase activity indicated below is substituted with isoleucine:

a protein having flavin-binding glucose dehydrogenase activity comprised of the amino acid sequence set forth in SEQ ID NO: 1, or

a protein having flavin-binding glucose dehydrogenase activity comprised of amino acids in which one or a plurality of amino acids other than the amino acid residue at the position corresponding to the leucine residue at position 391 in the amino acid sequence of SEQ ID NO: 1 has been deleted, substituted or added.

(10) An FAD-GDH in the form of a modified protein wherein the amino acid at the position corresponding to the leucine residue at position 557 in an amino acid sequence composing a protein having FAD-GDH activity indicated below is substituted with valine:

a protein having FAD-GDH activity comprised of the amino acid sequence set forth in SEQ ID NO: 1, or

a protein having FAD-GDH activity comprised of amino acids in which one or a plurality of amino acids other than the amino acid residue at the position corresponding to the leucine residue at position 557 in the amino acid sequence of SEQ ID NO: 1 has been deleted, substituted or added.

(11) An FAD-GDH wherein the amino acids at positions corresponding to the amino acid sequence represented by SEQ ID NO: 1, an amino acid sequence having a sequence identity of 70% or more with the amino acid sequence represented by SEQ ID NO: 1, or an amino acid sequence in which one or a plurality of amino acids in the amino acid sequence represented by SEQ ID NO: 1 or an amino acid sequence having a sequence identity of 70% or more with the amino acid sequence represented by SEQ ID NO: 1 have been deleted, substituted or added, are any of the amino acid residues set forth below:

the amino acid at the position corresponding to asparagine at position 66 in the amino acid sequence set forth in SEQ ID NO: 1 is tyrosine, and the amino acid at the position corresponding to asparagine at position 68 is glycine,

the amino acid at the position corresponding to cysteine at position 88 in the amino acid sequence set forth in SEQ ID NO: 1 is alanine, the amino acid at the position corresponding to asparagine at position 66 is tyrosine, and the amino acid at the position corresponding to asparagine at position 68 is glycine,

the amino acid at the position corresponding to cysteine at position 88 in the amino acid sequence set forth in SEQ ID NO: 1 is alanine, arid the amino acid at the position corresponding to threonine at position 158 is histidine,

the amino acid at the position corresponding to cysteine at position 88 in the amino acid sequence set forth in SEQ ID NO: 1 is alanine, and the amino acid at the position corresponding to glutamine at position 233 is arginine, or

the amino acid at the position corresponding to cysteine at position 88 in the amino acid sequence set forth in SEQ ID NO: 1 is alanine, the amino acid at the position corresponding to leucine at position 557 is valine, and the amino acid at the position corresponding to serine at position 559 is lysine.

(12) The FAD-GDH described in any of (1) to (11) above, provided with the properties described in (I) and/or (II) below:

(I) having a residual activity rate of 50% or more following heat treatment for 15 minutes at pH 7.0 and 40° C.;

(II) having a ratio of reactivity with D-xylose to reactivity with D-glucose (Xyl/Gle(%)) of 2% or less; and/or

(III) having a specific activity following introduction of a mutation of 60% or more in comparison with specific activity prior to introduction of a mutation.

(13) An FAD-GDH gene encoding the FAD-GDH described in any of (1) to (12) above.

(14) Recombinant DNA comprising the FAD-GDH gene described in (13) above inserted into vector DNA.

(15) A host cell introduced with the recombinant DNA described in (14) above.

(16) A method for producing FAD-GDH, comprising the following steps:

a step for culturing the host cell described in (15) above,

a step for expressing an FAD-GDH gene contained in the host cell, and

a step for isolating the FAD-GDH from the culture.

(17) A glucose measurement method using the FAD-GDH described in any of (1) to (12) above.

(18) A glucose assay kit containing the FAD-GDH described in any of (1) to (12) above.

(19) A glucose sensor containing the FAD-GDH described in any of (1) to (12) above.

### EFFECTS OF THE INVENTION

According to the present invention, FAD-GDH can be provided that has heat stability.

### BEST MODE FOR CARRYING OUT THE INVENTION

(Action Principle of FAD-GDH of Present Invention and Method for Measuring Activity)

The FAD-GDH of the present invention catalyzes a reaction that forms glucono-δ-lactone by oxidizing the hydroxyl group of glucose in the presence of an electron acceptor.

This action principle can be used to measure the activity of the FAD-GDH of the present invention by using the following system, for example, that uses phenazine methosulfate (PMS) and 2,6-dichloroindophenol (DCIP) as electron acceptors.

D-glucose PMS (oxidized form) →D-glucono-δ-lactone PMS (reduced form) (Reaction 1)

PMS (reduced form) DCIP (oxidized form)→PMS (oxidized form)+DCIP (reduced form) (Reaction 2)

In Reaction 1, PMS (reduced form) is formed accompanying oxidation of glucose. DCIP is then reduced accompanying oxidation of PMS (oxidized form) due to progression of the subsequent Reaction 2. By detecting the degree of disappearance of this DCIP (oxidized form) as an amount of change in optical absorbance at a wavelength of 600 nm, enzyme activity can be determined based on this amount of change.

Activity of the FAD-GDH of the present invention can be measured in accordance with the procedure indicated below. 2.05 mL of 100 mM phosphate buffer (pH 7.0), 0.6 mL or 1 M D-glucose solution and 0.15 mL of 2 mM DCIP solution are mixed and then warmed for 5 minutes at 37° C. Next, 0.1 mL of 15 mM PMS solution and 0.1 mL of enzyme sample solution are added to initiate the reaction. Optical absorbance is measured at the start of the reaction and over time, the amount of the decrease in optical absorbance at 600 nm (ΔA600) per minute accompanying progression of the enzyme reaction is determined, and GDH activity is calculated in accordance with the equation indicated below. At this time, 1 U of GDH activity is defined as the amount of enzyme that reduces 1 μmol of DCIP in one minute in the presence of D-glucose at a concentration of 200 mM at 37° C.

$$GDH activity ( U \text{/} mL ) = \frac{- ( Δ A 600 - Δ A {600}_{blank} ) \times 3.0 \times df}{16.3 \times 0.1 \times 1.0} \qquad [ Equation 1 ]$$

Furthermore, the value of 3.0 in the equation represents the amount of the reaction reagent and enzyme reagent (mL), 16.3 represents the millimolar molecular extinction coefficient (cm²/μmol), 0.1 represents the amount of the enzyme solution (mL), 1,0 represents the cell path length (cm), Δ600_{blank} represents the amount of decrease in optical absorbance per minute at 600 nm in the case of starting the reaction by adding the buffer used for enzyme dilution instead of the enzyme sample solution, and df represents the dilution factor.

(Amino Acid Sequence of FAD-GDH of Present Invention)

The FAD-GDH of the present invention is characterized by being comprised of the amino acid sequence represented by SEQ ID NO: 1, an amino acid sequence having a high degree of sequence identity with that amino acid sequence, such as preferably 70% or more, more preferably 75% or more, even more preferably 80% or more, still more preferably 85% or more, even more preferably still 90% or more, and most preferably 95% or more, or an amino acid sequence in which one or a plurality of amino acids in that amino acid sequence have been deleted, substituted or added, and having one or more amino acid substitutions at positions corresponding to the amino acids selected from among positions 66, 68, 88, 158, 233, 385, 391 and 557 in the amino acid sequence set form in SEQ ID NO: 1.

Preferably, an amino acid substitution at the position corresponding to the aforementioned position 66 in the FAD-GDH of the present invention is a substitution in which the amino acid at the position corresponding to the aforementioned position 66 is substituted with tyrosine, an amino acid substitution at the position corresponding to position 68 is a substitution in which the amino acid at the position corresponding to the aforementioned position 68 is substituted with glycine, an amino acid substitution at the position corresponding to position 88 is a substitution in which the amino acid at the position corresponding to the aforementioned position 88 is substituted with alanine, an amino acid substitution at the position corresponding to position 158 is a substitution in which the amino acid at the position corresponding to the aforementioned position 158 is substituted with histidine, an amino acid substitution at the position corresponding to position 233 is a substitution in which the amino acid at the position corresponding to the aforementioned position 233 is substituted with arginine, an amino acid substitution at the position corresponding to position 385 is a substitution in which the amino acid at the position corresponding to the aforementioned position 385 is substituted with threonine, an amino acid substitution at the position corresponding to position 391 is a substitution in which the amino acid at the position corresponding to the aforementioned position 391 is substituted with isoleucine, and an amino acid substitution at the position corresponding to position 557 is a substitution in which the amino acid at the position corresponding to the aforementioned position 557 is substituted with valine. Furthermore, in SEQ ID NO: 1, the amino acid at position 66 not having a substitution of the present invention is asparagine, the amino acid at position 68 is asparagine, the amino acid at position 88 is cysteine, and the amino acid at position 158 is threonine, the amino acid at position 233 is glutamine, and the amino acid at position 557 is leucine.

In the FAD-GDH of the present invention, more preferable examples thereof include multiple mutants having a plurality of combinations of the aforementioned substitutions. Double mutants combining two of the aforementioned substitutions, triple mutants combining three of the aforementioned substitutions and multiple mutants combining a large number of the aforementioned substitutions are included in the present invention. Accumulation of these mutations makes it possible to create FAD-GDH having more improved heat stability.

In addition, in creating multiple mutants as described above, substitutions at positions other than the positions of each of the types of substitutions described above can also be combined. In the case of introducing a single substitution, the position of the substitution may be that which does not demonstrate a remarkable effect in the manner of those at the aforementioned substitution sites, or may be that which demonstrates a synergistic effect as a result of introducing in combination with the aforementioned substitution sites.

In addition, the FAD-GDH of the present invention may also arbitrarily combine a mutation that improves substrate specificity or a known mutation for the purpose of demonstrating a different effect, such as an effect that improves resistance to pH or a specific substance, in addition to a mutation that improves heat stability as described above. Even in cases in which a different type of mutation has been combined, such FAD-GDH is also included in the present invention provided it is able to demonstrate the effect of the present invention.

As will be subsequently described, the FAD-GDH of the present invention can be obtained by, for example, first acquiring a gene that encodes an amino acid sequence similar to the amino acid sequence of SEQ ID NO: 1 by an arbitrary method, and then introducing an amino acid substitution at any position that is equivalent to a prescribed position of SEQ ID NO: 1.

Examples of methods used to introduce a target amino acid substitution include methods in which a mutation is introduced randomly and methods in which a site-directed mutation is introduced at a presumed position. Examples of the former methods include the Error-Prone PCR Method (Techniques, 1, 11-15 (1989)) and methods using XL1-Red competent cells, in which errors easily occur during plasmid replication and which are susceptible to the occurrence of modifications during cell proliferation (Stratagene Corp.). In addition, examples of the latter methods include methods consisting of constructing a three-dimensional structure based on a crystal structure analysis of a target protein, selecting an amino acid predicted to yield a target effect based on that information, and introducing a site-directed mutation using the commercially available Quick Change Site-Directed Mutagenesis Kit (Stratagene Corp.) and the like. Alternatively, another example of the latter methods is a method consisting of selecting an amino acid predicted to yield a target effect by using the three-dimensional structure of a known protein having a high degree of homology with a target protein and introducing a site-directed mutation.

In addition, the “position corresponding to the amino acid sequence of SEQ ID NO: 1” referred to here, for example, refers to the same position in that alignment in the case of aligning the amino acid sequence of SEQ ID NO:1 with another FAD-GDH having an amino acid sequence having sequence identity with SEQ ID NO: 1 (preferably 70% or more, more preferably 75% or more, even more preferably 80% or more, still more preferably 85% or more, even more preferably still 90% or more, and most preferably 95% or more). Furthermore, amino acid sequence identity can be calculated by a program such as the GENETYX-Mac maximum matching and search homology programs (Software Development Corp.) or the DNASIS Pro maximum matching and multiple alignment programs (Hitachi Software Engineering Co., Ltd.).

In addition, an example of a method for specifying the “position corresponding to the amino acid sequence of SEQ ID NO: 1” referred to here, for example, can be carried out by comparing amino acid sequences using a known algorithm such as the Lipman-Pearson method, and imparting maximum identity to conserved amino acid residues present in the amino acid sequence of FAD-GDH. Aligning FAD-GDH amino acid sequences using such methods makes it possible to determine the positions of corresponding amino acids in each of the FAD-GDH sequences regardless of the presence of insertions or deletions in the amino acid sequences. Corresponding positions are thought to be present at the same positions in three-dimensional structures, and can be assumed to have similar effects with respect to substrate specificity of the target FAD-GDH.

Although the FAD-GDH of the present invention is presumed to have various variations within the range of the aforementioned identity,.these various FAD-GDHs can all be included in the FAD-GDH of the present invention provided their enzymological properties are similar to the FAD-GDH of the present invention described in the present description. FAD-GDH having such an amino acid sequence demonstrates high substrate specificity and has adequate heat stability, thereby making it industrially useful.

In addition, it is important in the FAD-GDH of the present invention that the amino acid at the position corresponding to the aforementioned position 66 is tyrosine, the amino acid at the position corresponding to position 68 is glycine, the amino acid at the position corresponding to position 88 is alanine, the amino acid at the position corresponding to position 158 is histidine, the amino acid at the position corresponding to position 233 is arginine, the amino acid at the position corresponding to position 385 is threonine, the amino acid at the position corresponding to position 391 is isoleucine, or the amino acid at the position corresponding to position 557 is valine, while it is not important as to whether or not they are the result of an artificial substitution procedure. For example, in the case of using a protein such as the protein as set forth in SEQ ID NO: 1, in which the amino acids at the aforementioned positions are originally different from residues desired in the present invention, as a starting substance followed by introducing a desired substitution therein using a known technology, the desired amino acid residues are introduced by substitution. In contrast, in the case of acquiring a desired protein by a known total peptide synthesis, in the case of synthesizing an entire gene sequence so as to encode a protein having a desired amino acid sequence and acquiring a desired protein on the basis thereof or in the case of a natural protein found to originally have such a sequence therein, the FAD-GDH of the present invention can be obtained without having to go through a step consisting of artificial substitution.

(Improvement of Heat Stability in FAD-GDH of Present Invention)

Improvement of heat resistance in the present invention is evaluated under conditions described in the activity measurement method and heat stability measurement method described in the present description. Furthermore, the pH during heat treatment is 7.0 in the present description, because the FAD-GDH of the present invention was developed for the purpose of measuring glucose in blood (blood glucose level) and the pH of the blood is in the vicinity of neutrality. Evaluating under conditions approximating actual use in this manner makes it possible to acquire a useful enzyme.

The FAD-GDH of the present invention is characterized in that residual activity following heat treatment for 15 minutes at pH 7.0 and 40° C. under the reaction conditions described in the activity measurement method and thermal stability measurement method described in the present description is 50% or more, preferably 60% or more and more preferably 70% or more.

A more preferable FAD-GDH of the present invention is characterized in that residual activity following heat treatment for 15 minutes at pH 7.0 and 45° C. under the reaction conditions described in the activity measurement method and thermal stability measurement method described in the present description is 10% or more, 30% or more, preferably 50% or more and more preferably 70% or more.

In addition, in addition to improving heat stability as previously described, the FAD-GDH of the present invention is preferably also provided with performance more suitable for actual use with respect to other enzymatic properties as well. For example, the ratio of reactivity with D-xylose to reactivity with D-glucose (Xyl/(Glc(%)) and/or the ratio of reactivity with maltose to reactivity with D-glucose (Mal/Glc(%)) are preferably 2% or less. For example, specific activity is maintained at preferably 60% or more, more preferably at 65% or more, even more preferably at 70% or more, still more preferably at 75% or more, even more preferably at 80% or more, still more preferably at 85% or more and even more preferably at 90% or more in comparison with prior to introduction of a prescribed mutation. For example, the Km value is preferably 100 mM or less and preferably 90 mM or less.

(Acquisition of Gene Encoding FAD-GDH of Present Invention)

A genetic engineering technique is preferably used to efficiently acquire the FAD-GDH of the present invention. An ordinary, commonly used genetic cloning method may be used to acquire a gene encoding the FAD-GDH of the present invention (to be referred to as FAD-GDH gene). For example, in order to acquire the FAD-GDH of the present invention by using a known FAD-GDH as a starting substance followed by modification thereof, chromosomal DNA or mRNA can be extracted from known microbial cells or various other cells having the ability to produce FAD-GDH according to an ordinary method such as the method described in Current Protocols in Molecular Biology, Wiley Interscience (1989)). Moreover, cDNA can be synthesized by using mRNA as a template. A chromosomal DNA or cDNA library can then be prepared using chromosomal DNA or cDNA obtained in this manner.

Next, DNA containing the total length of a target FAD-GDH gene can be obtained by amplifying DNA containing target gene fragments encoding FAD-GDH having high substrate specificity and then linking these DNA fragments by a method in which a suitable probe DNA is synthesized based on amino acid sequence information of a known FAD-GDH followed by using this probe DNA to select an FAD-GDH gene having high substrate specificity from a chromosomal DNA or cDNA library, or a suitable polymerase chain reaction method (PCR method) such as 5′RACE or 3′RACE by preparing suitable primer DNA based on the aforementioned amino acid sequence.

A method consisting of introducing a mutation into a gene encoding FAD-GDH serving as a starting substance and then selecting FAD-GDH using enzymological properties of FAD-GDH expressed from various mutant genes as indices can be used as a method for acquiring the FAD-GDH of the present invention having superior heat stability by using a known FAD-GDH as a starting substance.

Any known method corresponding to an intended mutant form can be carried out for mutation treatment on the FAD-GDH gene serving as the starting substance. Namely, a widely used method can he used, such as a method consisting of contacting a chemical agent serving as a mutagen with an FAD-GDH gene or recombinant DNA incorporating that gene and allowing it to act thereon, an ultraviolet radiation method, a genetic engineering method or a method that employs a genetic engineering technique.

Examples of chemical agents used as mutagens in the aforementioned mutation treatment include hydroxylamine, N-methyl-N′-nitro-N-nitrosoguanidine, nitrous acid, sulfurous acid, hydrazine, formic acid and 5-bromouracil.

Conditions corresponding to the type of chemical agent used and the like can be employed for the various conditions for this contact and action, and there are no particular limitations thereon provided a desired mutation can actually he induced in an FAD-GDH gene derived from a Mucor species. Normally, a desired mutation can be induced by contacting and allowing to act for 10 minutes or longer, and preferably for 10 minutes to 180 minutes, at a concentration of the aforementioned chemical agent of 0.5 M to 12 M and at a reaction temperature of 20° C. to 80° C. In the case of irradiating with ultraviolet light as well, irradiation can be carried out in accordance with an ordinary method as previously described (Chemistry Today, 24-30, Jun. 1989).

A technique known as site-specific mutagenesis can typically be used as a method that employs a genetic engineering technique. Examples thereof include the Kramer method (Nucleic Acids Res., 12, 9441 (1984); Methods Enzymol., 154, 350 (1987); Gene, 37, 73 (1985)), the Eckstein method (Nucleic Acids Res., 13, 8749 (1985); Nucleic Acids Res., 13, 8765 (1985); Nucleic Acids Res., 14, 9679 (1986)), and the Kunkel method (Proc. Natl. Acad. Sci. U.S.A., 82, 488 (1985); Methods Enzymol., 154, 367 (1987)). Specific examples of methods for transforming a base sequence present in DNA include the use of a commercially available kit (such as the Transformer Mutagenesis Kit (Clontech Laboratories, Inc.), ExOIII/Mung Bean Deletion Kit (Stratagene Corp.) or Quick Change Site-Directed Mutagenesis Kit (Stratagene Corp.)).

In addition, a technique commonly known as a polymerase chain reaction also be used (Technique, 1, 11 (1989)).

Furthermore, in addition to the aforementioned genetic modification methods, a modified FAD-GDH gene having desired superior thermal stability can be synthesized directly by an organic synthesis method or enzymatic synthesis method.

The Multi-Capillary DNA Analysis System CEQ2000 (Beckman Coulter Inc.), for example, may be used in the case of determining or confirming the DNA base sequence of the FAD-GDH gene of the present invention selected according to any of the methods described above.

(Examples of Naturally-Occurring FAD-GDH Serving as

Source of FAD-GDH of Present Invention)

The FAD-GDH of the present invention can also be acquired by modifying a known FAD-GDH. Preferable examples of known microorganisms serving as sources of FAD-GDH include microorganisms classified as members of the subphylum Mueoromyeotina, preferably members of the class Mucoromycetes, more preferably members of the order Mucorales, and even more preferably members of the family Mucoraceae. Specific examples include FAD-GDH derived from Mucor species, Absidia species, Actinotnucor species and Circinella species.

Specific preferable examples of microorganisms classified as Mucor species include Mucor prainii, Mucor javanicus, Mucor circinelioides f. cirinelloides, Mucor guilliermondii, Mucor hiemalis f. silvaticus, Mucor subtilissimus and Mucor dimorphosporous. More specifically, examples include the Mucor prainii, Mucor javanicus, and Mucor circinelloides f. circinelloides described in Patent Document 5, and Mucor guilliermondii NBRC9403, Mucor hiemalis f. silvaticus NBRC6754, Mucor subtilissimus NBRC6338, Mucor RD056860 and Mucor dimorphosporous NBRC5395 Specific preferable examples of microorganisms classified as Absidia species include Absidia cylindrospora and Absidia hyalospora. More specifically, examples include the Absidia cylindrospora and Absidia hyalospora described in Patent Document 5. Specific preferable examples of microorganisms classified as .Actinomucor species include Actinomucor elegans. More specific examples include the Actinomucor elegans described in Patent Document 5. Specific preferable examples of microorganisms classified as Circinella species include Circinella minor, Circinella mucoroides, Circinella muscae, Circinella rigida, Circinella simplex and Circinella umbellata. More specific examples include Circinella minor NBRC6448, Circinella mucoroides NBRC4453, Circinella muscae NBRC6410, Circinella rigida NBRC6411, Circinella simplex NBRC6412, Circinella umbellate NBRC4452, Circinella umbellata NBRC5842, Circinella RD055423 and Circinella RD055422. Furthermore, NBRC strains and RD strains refer to stock strains of the Patent Microorganisms Depositary Center of the National Institute of Technology and Evaluation.

(Vectors and Host Cells Inserted with FAD-GDH Gene of Present Invention)

PAD-GDH gene of the present invention obtained in the manner described above can be incorporated in a vector such as a bacteriophage, cosmid or plasmid used to transform prokaryotic cells or eukaryotic cells in accordance with ordinary methods followed by transforming or transducing host cells corresponding to each vector in accordance with ordinary methods.

Examples of prokaryotic host cells include microorganisms belonging to the genus Escherichia such as Escherichia coli strain K-12, Escherichia coli BL21(DE3), Escherichia coli JM109, Escheriehia coli DH5α, Escherichia coli W3110 or Escherichia coli C600 (all available from Takara Bio Inc.). These cells can then be transformed or transduced to obtain host cells introduced with DNA (transformants). A method for transferring recombinant DNA in the presence of calcium ions can be used to transfer a recombinant vector into such host cells in the case, for example, the host cells are microorganisms belonging to the genus Escherichia. Moreover, electroporation may also be used. Commercially available competent cells (such as ECOS Competent Escherichia coli BL21(DE3), Nippon Gene Co., Ltd.) may also be used.

An example of eukaryotic host cells is yeast. Examples of microorganisms classified as yeast include yeast belonging to the genus Zygosaccharomyces, genus Saccharomyces, genus Pichia and genus Candida. Inserted genes may contain marker genes for enabling selection of transformed cells. Examples of marker genes include genes that complement the nutritional requirements of the host in the manner of URA3 or TRP 1. In addition, the inserted gene preferably also contains a promoter, enabling a gene of the present invention to be expressed in host cells, or other control sequences (such as an enhancer sequence, terminator sequence or polyadenylation sequence). Specific examples of promoters include the GAL1 promoter and ADH1 promoter. Although a known method such as a method using lithium acetate (Methods Mol. Cell. Biol., 5, 255-269 (1995)) or electroporation (J. Microbiol. Methods, 55, 481-484 (2003)) can be preferably used to transform yeast, the method used is not limited thereto, but rather transformation may be carried out using various types of arbitrary techniques such as the spheroplast method or glass bead method.

Other examples of eukatyotic host cells include mold cells in the manner of Aspergillus species and Tricoderma species. The inserted gene preferably contains a promoter, enabling a gene of the present invention to be expressed in host cells (such as the tef1 promoter), and other control sequences (such as a secretion signal sequence, enhancer sequence, terminator sequence or polyadenylation sequence). In addition, the inserted gene may also contain a marker gene such as niaD or pyrG for enabling the selection of transformed cells. Moreover, the inserted gene may also contain a homologous recombination domain for inserting into an arbitrary chromosome site. A known method such as a method using polyethylene glycol and calcium chloride following protoplast formation (Mol. Gen. Genet., 218, 99-104 (1989)) can be preferably used to transform molds.

(Production of FAD-GDH of Present Invention)

The FAD-GDH of the present invention may be produced by culturing host cells that produce the FAD-GDH of the present invention acquired in the manner described above, expressing the FAD-GDH gene contained in the host cells, and then isolating FAD-GDH from the culture.

Examples of media used to culture the aforementioned host cells include that obtained by adding one or more types of inorganic salts such as sodium chloride, potassium dihydrogen phosphate, dipotassium hydrogen phosphate, magnesium sulfate, magnesium chloride, ferric chloride, fenic sulfate or manganese sulfate to one or more types of nitrogen sources such as yeast extract, tryptone, peptone, beef extract, corn stiplica or soybean or wheat bran exudate, and further suitably adding carbohydrate raw materials or vitamins and the like as necessary.

Although there are no particular limitations thereon, e initial pH of the medium can be adjusted to, for example pH 6 to 9.

Culturing may be carried out by aeration-agitation submerged culturing, shake culturing or static culturing and the like preferably for 4 hours to 24 hours at a culturing temperature of 10° C. to 42° C., and preferably about 25° C., and more preferably for 4 hours to 8 hours at about 25° C.

Following completion of culturing, the FAD-GDH of the present invention is harvested from the culture. An ordinary known enzyme harvesting means may be used. For example, microbial cells can be subjected to ultrasonic pulverization treatment or grinding treatment in accordance with ordinary methods, the enzyme can be extracted using a lytic enzyme such as lysozyme, or the enzyme can be expelled outside the microbial cells by lysing the cells by shaking or allowing to stand in the presence of toluene and the like. A crude FAD-GDH of the present invention is then obtained by filtering this solution, removing the solid fraction by centrifugal separation, removing nucleic acids with streptomycin hydrochloride, protamine sulfate or manganese sulfate and the like as necessary, followed by fractionating by addition of ammonium sulfate, alcohol or acetone and the like and harvesting the precipitate.

The crude FAD-GDH enzyme of the present invention can be further purified using any known means. A purified FAD-GDH enzyme preparation of the present invention can be obtained by, for example, suitably selecting a gel filtration method using Sephadex, Ultrogel or Biogel, an adsorption elution method using an ion. exchanger, an electrophoresis method using polyacrylamide gel, an adsorption elution method using hydroxyapatite, a precipitation method such as sucrose density gradient centrifugation, an affinity chromatography method, or a fractionation method using a molecular sieve membrane or hollow fiber membrane, or using a combination thereof.

(Method for Measuring Glucose Using FAD-GDH of Present Invention)

The present invention also discloses a glucose assay kit that contains the FAD-GDH of the present invention, and glucose in the blood (blood glucose level) can be measured using the FAD-GDH of the present invention.

The glucose assay kit of the present invention contains an amount of FAD-GDH modified in accordance with the present invention sufficient for at least one assay. Typically, the glucose assay kit of the present invention also contains a buffer, mediator and glucose standard solutions for preparing a calibration curve in addition to the modified FAD-GDH of the present invention. The modified FAD-GDH used in the glucose measurement method and glucose assay kit of the present invention can be provided in various forms, such as in the form of a freeze-dried reagent or dissolved in a suitable preservative solution.

Measurement of glucose concentration can be carried out in the manner indicated below, for example, in the case of a colorimetric glucose assay kit. A liquid or solid composition containing FAD-GDH an electron acceptor and one or more reaction accelerators in the form of substances selected from the group consisting of N-(2-acetamido)iminodiacetic acid (ADA), bis(2-hydroxyethyl)iminotris(hydroxymethyl)methane (Bis-Tris), sodium carbonate and imidazole is retained in the reaction layer of the glucose assay kit. Here, a pH buffer or coloring reagent is added as necessary. A sample containing glucose is then added thereto and allowed to react for a fixed period of time. During this time, optical absorbance corresponding to the maximum absorption wavelength of an electron acceptor that is discolored by reduction, or a pigment formed by polymerization as a result of accepting electrons from an electron acceptor, is monitored. Glucose concentration in the sample can be calculated on the basis of a calibration curve prepared in advance using glucose solutions having standard concentrations, from the rate of change in absorbance per unit time if using a rate method, or from the change in absorbance to the point all glucose in the sample has been oxidized if using the endpoint method.

In the case of using a mediator and coloring reagent able to be used in this method, glucose can be quantified by adding 2,6-dichlorophenolindophenol (DCPIP) as an electron acceptor followed by monitoring the decrease in absorbance at 600 nm. In addition, glucose concentration can be calculated by adding an electron acceptor in the form of phenazine methosulfate (PMS) and a coloring reagent in the form of nitrotetrazolium blue (NTB) followed by determining the amount of diformazan formed by measuring absorbance at 570 nm. Furthermore, it goes without saying that the electron acceptors and coloring reagents used are not limited thereto.

(Glucose Sensor Containing FAD-GDH of Present Invention)

The present invention also discloses a glucose sensor that uses the FAD-GDH of the present invention. An electrode such as a carbon electrode, gold electrode or platinum electrode is used for the electrode, and the FAD-GDH of the present invention is immobilized on this electrode. Examples of immobilization methods include a method using a crosslinking agent, a method consisting of enclosing in a polymer matrix, a method consisting of coating with a dialysis membrane and methods using a photocrosslinkable polymer, electrically conductive polymer and oxidation-reduction polymer, or the FAD-GDH may be immobilized in a polymer together with an electrode mediator represented by fenocene or a derivative thereof, immobilized by adsorbing to the electrode, or a combination of these methods may be used. Typically, the FAD-GDH of the present invention is immobilized on a carbon electrode using glutaraldehyde followed by treating with a reagent having an amino group to block the glutaraldehyde.

Glucose concentration can be measured in the manner indicated below. A buffer is placed in a thermostatic cell and maintained at a constant temperature. Potassium ferricyanide or phenazine methosulfate, for example, can be used for the mediator. The electrode having the modified FAD-GDH of the present invention immobilized thereon is used for the working electrode, and a counter electrode (such as a platinum electrode) and reference electrode (such as an Ag/AgCl electrode) are also used. A constant voltage is applied to the carbon electrode, and after the current has reached a steady state, a sample containing glucose is added followed by measurement of the increase in current. The glucose concentration in the sample can then be calculated in accordance with a calibration curve prepared with glucose concentrations having standard concentrations.

As a specific example thereof, 1.5 U of the FAD-GDH of the present invention are immobilized on a glassy carbon (GC) electrode and the response current value with respect to glucose concentration is measured. 1.8 ml of 50 mM potassium phosphate buffer (pH 6.0) and 0.2 ml of a 1 M aqueous solution of potassium hexacyanoferrate (III) (potassium ferricyanide) are added to an electrolysis cell. The GC electrode is connected to a BAS100B/W potentiostat (BAS Co., Ltd.) and the solution is stirred at 37° C. followed by applying a voltage of +500 my to the silver/silver chloride reference electrode. Glucose solutions having final concentrations of 5 mM, 10 mM, 20 mM, 30 mM, 40 mM and 50 mM are added to these systems followed by measurement of the steady-state current value for each addition. These current values are plotted versus known glucose concentrations (5 mM, 10 mM, 20 mM, 30 mM 40 mM, 50 mM) to prepare a calibration curve. As a result, glucose can be quantified with an enzyme-immobilized electrode using the FAD-binding glucose dehydrogenase of the present invention.

The following provides a more detailed explanation of the present invention through examples thereof. However, the technical scope of the present invention is not limited in any way by these examples.

### EXAMPLES

In the present invention, evaluations of heat stability and substrate specificity of modified FAD-GDH were carried out in accordance with the methods of the test example indicated below unless specifically mentioned otherwise.

### Test Example

(1) Preparation of Yeast Transformants Expressing Various Types of Modified FAD-GDH

A recombinant plasmid (pYES2C-Mp (wild type)) that encodes FAD-GDH acne derived from Mucor prainii (wild-type MpGDH gene) of SEQ ID NO: 2 was acquired in compliance with the method described in Patent Document 7.

PCR reactions were carried out under the conditions indicated below using KOD-Plus-(Toyobo Co., Ltd.) and synthetic nucleotides for introducing various amino acid substitutions using the resulting recombinant plastnid pYE2C-Mp as template.

In other words, 5 μl of 10×KOD-Plus-buffer, 5 μl of a dNTPs mixed solution prepared so that the concentration of each dNTP was 2 mM, 2 μl of 25 mM MgSO₄, 50 ng of pYE2C-Mp serving as template, 15 μmol of each of the synthetic oligonucleotides, and 1 unit of KOD-Plus—were added followed by adding sterilized water to a total volume of 50 μl to prepare a reaction solution. The prepared reaction solution was incubated for 2 minutes at 94° C. using a thermal cycler (Eppendorf AG) followed by repeating 30 cycles consisting of 15 seconds at 94° C., 30 seconds at 55° C. and 8 minutes at 68° C.

A portion of the reaction solution treated in the manner described above was subjected to electrophoresis with 1.0% agarose gel to confirm that about 8 kbp of DNA had been specifically amplified. The amplified DNA was treated with restrictase Dpnl (New England Biolabs Inc.) followed by carrying out transformation by mixing with competent cells of E. coli strain JM109 (Nippon Gene Co., Ltd.) in accordance with the protocol provided. Next, the acquired transformants were respectively applied to LB-amp agar media and cultured. The colonies that formed were inoculated into LB-amp culture broth, and subjected to shake culturing followed by isolating various types of plasmid DNA containing about 8 kbp of amplified DNA (such as pYE2C-Mp-N66Y/N68G or pYE2C-Mp-C88A in Example 1) in accordance with the protocol provided. Next, the base sequences of DNA encoding MpGDH gene in each of the plasmid DNA were determined using the Multi-Capillary DNA Analysis System CEQ2000 (Beekman Coulter Inc.), and amino acids were confirmed to be substituted at the prescribed positions in the amino acid sequence set forth in SEQ ID NO: 1. In this manner, yeast expression vectors were acquired that encode modified MpGDH having prescribed amino acid substitutions (modified types, such as pYE2C-Mp-N66Y/N68G or pYE2C-Mp-C88A in Example 1).

Subsequently, pYE2C-Mp (wild type) and various types of mutation-introduced pYES2C-Mp (modified types, such as pYE2C-Mp-N66Y/N68G or pYE2C-Mp-C88A in Example 1) were transformed in strain Inv-Sc (Invitrogen Corp.) using an S. cerevisiae transformation kit (Invitrogen Corp.) to respectively acquire yeast transformant, strain Sc-Mp (wild type) expressing wild-type MpGDH and yeast transformant strains Sc-Mp expressing various types of modified MpGDH (modified types, such as Sc-Mp-N66Y7N68G and Sc-Mp-C88A in Example 1).

(2) Evaluation of Heat Stability of Yeast-Expressed FAD-GDH

The yeast transformant strain Sc-Mp (wild type) and various yeast transformant strains Sc-Mp (modified type, such as Sc-Mp-N66Y/N68G and Sc-Mp-C88A in Example 1) were respectively cultured for 24 hours at 30° C. in 5 mL of pre-culturing broth (consisting of 0.67% (w/v) yeast nitrogen base without amino acids (Becton, Dickinson & Co.), 0.192% (w/v) yeast synthetic drop-out medium supplement without uracil (Sigma Corp.) and 2.0% (w/v) raffinose). Subsequently, 1 ml of pre-culturing broth was added to 4 mL of final culturing broth (consisting of 0.67% (w/v) yeast nitrogen base without amino acids, 0.192% (w/v) yeast synthetic drop-out medium supplement without uracil, 2.5% (w/v) D-galactose and 0.75% (w/v) raffinose) followed by culturing for 16 hours at 30° C. The culture broth was then centrifUged (10,000×g, 4° C., 3 minutes) to separate the microbial cells and culture supernatant, after which the culture supernatant was used to evaluate heat stability.

Heat stability of FAD-GDH was evaluated by first diluting the culture supernatant containing the FAD-ODH targeted for evaluation recovered in the manner described above with enzyme diluent (100 mM potassium phosphate buffer (pH 7.0)) to about 1 U/ml. Two of these enzyme solutions (0.1 ml each) were prepared and one was stored at 4° C. while the other was subjected to heat treatment for 15 minutes at 40° C.

Following heat treatment, the FAD-GDH activity of each sample was measured, and the activity value after treating for 15 minutes at 40° C. was calculated in the form of the “residual activity rate” based on a value of 100 for the enzyme activity in the solution stored at 4° C. This residual activity rate was used as an index for evaluation of heat resistance of each type of FDA-GDH.

As a result of evaluating heat stability of the wild-type MpGDH using the culture supernatant of strain Sc-Mp (wild type) expressing wild-type MpGDH, the residual activity rate following heat treatment of wild-type MpGDH for 15 minutes at 40° C. was 42.4%. Accordingly, heat stability of MpGDH can be judged to have improved in the case the residual activity rate following heat treatment of each type of modified MpGDH is higher than 42.4%.

(3) Evaluation of Substrate Specificity

Substrate specificity was evaluated using each type of yeast culture supernatant harvested in accordance with the method described in (2) above in the same manner as evaluation of heat stability. First, activity with respect to each substrate was measured by changing the substrate used in the aforementioned activity measurement method from D-glucose to a system containing the same molar concentration of maltose or D-xylose. The “ratio of reactivity with maltose to reactivity with D-glueose (Mal/Glc(%))” or the “ratio of reactivity with D-xylose to reactivity with D-glucose (Xyl/Glc(%))” was calculated from these values.

The values of (Mal/Glc(%)) and (Xyl/Glc(%)) of wild-typeMpGDH expressed in strain Sc-Mp (wild type) were 0.8% and 1.4%, respectively. This level of substrate specificity is extremely superior even in comparison with other conventionally known FAD-GDH, and is expected to enable accurate measurement of the target measured substance in the form of D-glucose.

### EXAMPLE 1

(Preparation of Various Modified MpGDH and Evaluation of Heat Stability)

PCR reactions were carried out on the combinations of synthetic nucleotides having the sequence ID numbers shown in Table 1 using pYE2C-Mp (wild type) as template plasmid in accordance with the method described in the previous test example. Next, E. coli strain JM109 was transformed using a vector containing amplified DNA, and by determining the base sequence of DNA encoding MpGDH in the plasmid DNA retained by the colonies that formed, recombinant plasmids in the form of pYE2C-Mp-N66Y/N68G, pYE2C-Mp-C88A, pYE2C-Mp-T158H, pYE2C-Mp-Q233R and pYE2C-Mp-L557V/S559K were acquired in which asparagine at position 66 of the amino acid sequence set forth in SEQ ID NO: 1 was substituted with tyrosine and asparagine at position 68 was substituted with glycine, cysteine at position 88 was substituted with alanine, threonine at position 158 was substituted with histidine, glutamine at position 233 was substituted with arginine, leucine at position 557 was substituted with valine and serine at position 559 was substituted with lysine, respectively.

Next, strain Inv-Sc was transformed and the acquired transformant strains (strain Sc-Mp-N66Y/N68G, strain Sc-Mp-C88A, strain Se-Mp-T158H, strain Sc-Mp-Q233R and strain Sc-Mp-1,557V/S559K) were cultured in accordance with section (2) of the test example using recombinant plasmids pYE2C-Mp-N66Y/N68G, pYE2C-Mp-C88A, pYE2C-Mp-T158H, pYE2C-Mp-Q233R and pYE2C-Mp-L557V/S559K encoding each modified type of MpGDH introduced with a site-directed mutation, followed by measuring the GDH activity in the culture supernatants.

Continuing, residual activity rate (%) after subjecting to heat treatment for 15 minutes at 40° C. and the ratio of reactivity with D-xylose to reactivity with D-glucose (Xyl/Glc(%)) were measured based on the procedures of (2) and (3) of the aforementioned test example using the culture supernatant of each of the aforementioned mutants for which GDH activity was confirmed.

Furthermore, in Table 1, for example, “C88A” means that C (Cys) at position 88 is substituted with A (Ala). In addition, “N66Y/N68G”, for example, means that N (Asn) at position 66 is substituted with Y (Tyr) and N (Asn) at position 68 is substituted with G (Gly), and the slash “/” means that this mutant has both substitutions.

TABLE 1

| | Primer | | |
|---|---|---|---|
| | Sequence | Residual Activity Rate | Xyl/Glc |
| Recombinant Plasmid | No. | (40° C., 15 min) (%) | (%) |
| pYE2C-Mp (wild type, | — | 42.4 | 1.4 |
| comparative example) | | | |
| pYE2C-Mp-N66Y/N68G | 3, 4 | 72.8 | 1.5 |
| (present invention) | | | |
| pYE2C-Mp-C88A | 5, 6 | 58.6 | 1.2 |
| (present invention) | | | |
| pYE2C-Mp-T158H | 7, 8 | 75.2 | 1.1 |
| (present invention) | | | |
| pYE2C-Mp-Q233R | 9, 10 | 73.5 | 1.8 |
| (present invention) | | | |
| pYE2C-Mp-L557V/S559K | 11, 12 | 52.0 | 1.6 |
| (present invention) | | | |

As shown in Table 1, the heat stability of FAD-GDH was confirmed to improve as a result of site-directed mutagenesis at position 66, 68, 88, 158, 233, 557 or 559 in the wild-type MpGDH of SEQ ID NO: 1, and more specifically, as a result of introducing a site-direction mutation of N66Y/N68G, C88A, T158H, Q233R or L557V/S559K.

Moreover, these FAD-GDH having improved heat stability were determined to also maintain high substrate specificity. Namely, modified enzymes having the heat stability-improving mutations of the present invention as shown in Table I were determined to not have a detrimental effect on substrate specificity of the wild-type FAD-GDH, and depending on the case, demonstrated substrate specificities that exceeded that of the wild-type enzyme.

### EXAMPLE 2

(Study on Combined Mutation Introduction)

Next, mutants were prepared having multiple mutations as shown in Example 2, and the effect of improving their heat stability was verified. More specifically, PCR reactions were carried out on the combinations of synthetic nucleotides having the sequence ID numbers shown in Table 2 using pYE2C-Mp-C88A as template plasmid in accordance with the method described in the aforementioned test example. Next, E. coli strain JM109 was transformed using a vector containing amplified DNA, and by determining the base sequence of DNA encoding MpGDH in the plasmid DNA retained by the colonies that formed, the multiple mutants indicated below were prepared that were characterized by having cysteine at position 88 substituted with alanine, and were also provided with a different amino acid substitution. More specifically, recombinant plasmids in the form of pYE2C-I′dp-C88A/N66Y/N68G, pYE2C-Mp-C88A/T158H, pYE2C-Mp-C88A/Q233R and pYE2C-Mp-C88A/L557V/S559K (were acquired that respectively encoded a triple mutation in which cysteine at position 88 of the amino acid sequence set forth in SEQ ID NO: 2 was substituted with alanine, asparagine at position 66 was substituted with tyrosine and asparagine at position 68 was substituted with glycine, a double mutant in which cysteine at position 88 was substituted with alanine, and threonine at position 158 was substituted with histidine, a double mutant in which cysteine at position 88 was substituted with alanine, and glutamine at position 233 was substituted with arginine, and a triple mutant in which cysteine at position 88 was substituted with alanine, leucine at position 557 was substituted with valine and serine at position 559 was substituted with lysine.

Next, strain Inv-Sc was transformed and the acquired transformant strains (strain Sc-Mp-C88A/N66Y/N68G, strain Sc-Mp-C88A/T158H, strain Sc-Mp-C88A/Q233R and strain Sc-Mp-C88A/L557V/S559K) were cultured in accordance with section (2) of the test example using recombinant plasmids (pYE2C-Mp-C88A/N66Y/N68G, pYE2C-Mp-C88A/T158H, pYE2C-Mp-C88A/Q233R and pYE2C-Mp-C88A/L557V/S559K) encoding each modified type of MpGDH introduced with a site-directed mutation, followed by measuring the GDH activity in the culture supernatants.

Continuing, residual activity rate (%) after subjecting to heat treatment for 15 minutes at 40° C., residual activity rate (%) after subjecting to heat treatment for 15 minutes at 45° C. and the ratio of reactivity with D-xylose to reactivity with D-glucose (Xyl/GlcM)) were measured based on the procedures of sections (2) and (3) of the aforementioned test example using the culture supernatant of each of the aforementioned mutants for which GDH activity was confirmed.

TABLE 2

| | Primer | Residual Activity | | |
|---|---|---|---|---|
| | Sequence | Rate (%) | | Xyl/Glc |
| Recombinant Plasmid | No. | 40° C. | 45° C. | (%) |
| pYE2C-Mp (wild type, | — | 42.4 | 0.0 | 1.4 |
| comparative example) | | | | |
| pYE2C-Mp-C88A | — | 58.6 | 2.0 | 1.2 |
| (present invention) | | | | |
| pYEC2-Mp-C88A/N66Y/N68G | 3, 4 | 74.7 | 12.5 | 1.3 |
| (present invention) | | | | |
| pYE2C-Mp-C88A/T158H | 7, 8 | 77.9 | 37.4 | 0.9 |
| (present invention) | | | | |
| pYE2C-Mp-C88A/Q233R | 9, 10 | 82.2 | 30.1 | 1.4 |
| (present invention) | | | | |
| pYE2C-Mp-C88A/L557V/ | 11, 12 | 63.4 | 4.1 | 1.4 |
| S559K (present invention) | | | | |

As shown in Table 2, heat resistance was confirmed to improve as a result of respectively combining the amino acid substitutions of N66Y/N68G, T158H, Q233R and L557V/S559K after having introduced C88A in the amino acid sequence of SEQ ID NO: 1. In particular, mutant C88A/N66Y/N68G retained a residual activity rate of 10% or more following heat treatment at 45° C., while mutant C88A/T158H and mutant C88A/Q233R retained residual activity rates of 30% or more following heat treatment at 45° C., and were particularly preferable multiple mutants.

Moreover, these multiple mutants were determined to also maintain or improve high substrate specificity, and substrate specificity of the wild type was detenuined to improve particularly in C88A/N66Y/N68G and C88A/T158H.

### EXAMPLE 3

(Study of Single Mutations)

Next, mutants were prepared having single mutations as shown in Example 3, and the effect of improving their heat stability was verified. More specifically, PCR reactions were carried out on the combinations of synthetic nucleotides having the sequence ID numbers shown in Table 3 using pYE2C-Mp (wild type) as template plasmid in accordance with the method described in the aforementioned test example. Next, E. coli strain JM109 was transformed using a vector containing amplified DNA, and by determining the base sequence of DNA encoding MpGDH in the plasmid DNA retained by the colonies that formed, recombinant plasmids in the form of pYE2C-Mp-N66Y, pYE2C-Mp-N68G, pYE2C-Mp-L391I, pYE2C-Mp-L557V, pYE2C-Mp-S559K and pYE2C-Mp-A385T were acquired that respectively encoded mutants in which asparagine at position 66 of the amino acid sequence set forth in SEQ ID NO: 1 was substituted with tyrosine, asparagine at position 68 was substituted with glycinc, leucine at position 391 was substituted with isoleucine, leucine at position 557 was substituted with valine, serine at position 559 was substituted with lysine and alanine at position 385 was substituted with threonine.

Next, strain InvSc was transformed and the acquired transformant strains (strains Sc-Mp-N66Y, Sc-Mp-N68G, Sc-Mp-L391I, Sc-Mp-L557V, Sc-Mp-S559K and Sc-Mp-A385T) were cultured in accordance with section (2) of the test example using recombinant plasmids (pYE2C-Mp-N66Y, pYE2C-Mp-N68G, pYE2C-Mp-L391I, pYE2C-Mp-L557V, pYE2C-Mp-S559K and pYE2C-Mp-A385T) encoding each modified type of MpGDH introduced with a site-directed mutation, followed by measuring the GDH activity in the culture supernatants.

Continuing, residual activity rate (%) after subjecting to heat treatment for 15 minutes at 40° C., residual activity rate (%) after subjecting to heat treatment for 15 minutes at 45° C., and the ratio of reactivity with D-xylose to reactivity with D-glucose (Xyl/Glc(%)) were measured based on the procedures of sections (2) and (3) of the aforementioned test example using the culture supernatant of each of the aforementioned mutants for which GDH activity was confirmed.

TABLE 3

| | Primer | | |
|---|---|---|---|
| | Sequence | Residual Activity Rate | Xyl/Glc |
| Recombinant Plasmid | No. | (40° C., 15 min) (%) | (%) |
| pYE2C-Mp (wild type) | — | 42.4 | 1.4 |
| pYE2C-Mp-N66Y/N68G | 3, 4 | 72.8 | 1.5 |
| pYE2C-Mp-N66Y | 13, 4 | 57.0 | 1.5 |
| pYE2C-Mp-N68G | 14, 4 | 26.1 | 1.3 |
| pYE2C-Mp-L391I | 15, 16 | 50.3 | 1.7 |
| pYE2C-Mp-L557V/S559K | 11, 12 | 52.0 | 1.6 |
| pYE2C-Mp-L557V | 17, 12 | 50.8 | 1.6 |
| pYE2C-Mp-S559K | 18, 12 | 42.5 | 1.4 |
| pYE2C-Mp-A385T | 19, 20 | 75.6 | 1.2 |

As shown in Table 3, heat resistance was confirmed to improve as a result of respectively introducing the amino acid substitutions of N66Y, L391I, L557V and A385T in the amino acid sequence of SEQ ID NO: 1. In addition, although heat resistance decreased in the case of introduction of a single mutation in the single mutation of N68G, combining with N66Y was confirmed to have the effect of improving heat resistance.

Moreover, since the values of Xyl/Glc (%) were 2% or less in all of these mutants, high substrate specificity was confirmed to be maintained or improved.

### EXAMPLE 4

(Measurement of Specific Activity U/A280 in each Mutant)

Activity per unit protein weight (specific activity) was measured for each of the mutants acquired in Examples 1 and 3 (NG6Y/N68G, C88A, T158H, Q233R, L557V/S559K, L391I and A385T). More specifically, the procedure indicated below was carried out. After concentrating yeast culture supernatants of each mutant acquired in the same manner as Examples 1 and 3 with a centrifugal filter unit (Amicon Ultra 10K, Merck Millipore Corp.), the supernatant was replaced with 20 mM potassium phosphate buffer (pH 6M). Since there were hardly any bands other than that corresponding to FAD-GDH observed when the concentrated yeast culture supernatants were subjected to SDS-PAGE for confirmation, the yeast culture supernatants were determined to contain hardly any contaminating proteins. Accordingly, protein concentration was measured using the concentrated yeast culture supernatants based on GDH activity and optical absorbance at 280 nm (A280), and the specific activity (U/A280) of each mutant was measured. Subsequently, the ratio of specific activity of each mutant was calculated as “relative specific activity” based on a value of 100 for specific activity prior to mutation introduction (wild type) measured in the same manner, and that value was used to evaluate specific activity. In other words, when relative specific activity is greater than 100, specific activity can be considered to have improved from that prior to the introduction of the mutation, and when relative specific activity is less than 100, specific activity can be considered to have decreased from that prior to mutation introduction. Furthermore, since the relative specific activity of mutant C88A, which was calculated from specific activity measured using the crude enzyme solution of the present method, was 125, while the relative specificity of C88A after having been purified with the Superdex 200 1.0/300GL column (GE Healthcare Biosciences Inc.) was 119, the value for relative specific activity as calculated according to the present method was judged to correlate with the value for relative specific activity measured using the purified enzyme.

In addition, the relative specific activities of Mucor-derived FAD-(3DH mutants V232E, T387A. and I545T described in Patent Document 7 as demonstrating improved heat resistance were also measured in the same manner as described above, and heat stability and substrate specificity were evaluated in accordance with sections (2) and (3) of the test example.

TABLE 4

| | | Residual | |
|---|---|---|---|
| | Relative | Activity | |
| | Specific | Rate (40° C., | Xyl/Glc |
| Recombinant Plasmid | Activity | 15 min) (%) | (%) |
| pYE2C-Mp (wild type, comparative | 100 | 42.4 | 1.4 |
| example) | | | |
| pYE2C-Mp-N66Y/N68G (present | 93 | 72.8 | 1.5 |
| invention) | | | |
| pYE2C-Mp-C88A (present invention) | 125 | 58.6 | 1.2 |
| pYE2C-Mp-T158H (present invention) | 70 | 75.2 | 1.1 |
| pYE2C-Mp-Q233R (present invention) | 91 | 73.5 | 1.8 |
| pYE2C-Mp-L557V/S559K (present | 105 | 52.0 | 1.6 |
| invention) | | | |
| pYE2C-Mp-L391I (present invention) | 92 | 50.3 | 1.7 |
| pYE2C-Mp-A385T (present invention) | 79 | 75.6 | 1.2 |
| pYE2C-Mp-V232E (Pat. Doc. 7, | 109 | 55.8 | 2.9 |
| comparative example) | | | |
| pYE2C-Mp-T387A (Pat. Doc. 7, | 57 | 89.3 | 0.9 |
| comparative example) | | | |
| pYE2C-Mp-I545T (Pat. Doc. 7, | 55 | 91.3 | 0.9 |
| comparative example) | | | |

As shown in Table 4, although mutants demonstrating improved heat resistance described in Patent Document 7 in the form of T387A and I545T maintain a high level of substrate specificity, demonstrating a value of Xyl/Glc (%) of lower than 2, since relative specific activity is below 60%, specific activity was determined to decrease considerably. In addition, although the mutant demonstrating improved heat resistance described in Patent Document 7 in the form of V232E maintained a high specific activity of greater than 100, the value of Xyl/Glc (%) was higher than 2%, and high substrate specificity was determined to be impaired.

In contrast, the mutants demonstrating improved heat resistance of the present invention in the form of N66Y/N68G, C88A, T158H, Q233R, L557V/S559K, L391I and A385T all retained relative specific activities of 60% or hialier, and as previously described, since they all demonstrated values for Xyl/Glc (VD) of lower than 2%, they were determined to maintain a high level of substrate specificity.

As has been described above, the mutants of the present invention were determined to demonstrate improved heat stability in comparison with enzymes prior to introduction of a mutation and have adequate heat stability, FAD-GDH having this property makes it possible to reduce the amount of enzyme used and prolong shelf life in the case of using to produce assay reagents or assay kits due to the low degree of enzyme thermal deactivation, and is expected to be able to provide a measurement method, measurement reagent, measurement kit and sensor that are more practical than measurement methods and measurement reagents using known enzymes for glucose measurement. In particular, the FAD-GDH of the present invention demonstrating superior heat stability is considered to be extremely useful in processes for producing chips for use in blood glucose sensors and the like, which can be presumed to be subjected to heat drying treatment.

Furthermore, as disclosed in the present description, the mutant enzymes demonstrating improved heat stability of the present invention were determined to include those capable of accurately measuring D-clucose values, even under conditions of contamination by sugar compounds such as D-xyl.ose, as a result of being provided with high substrate specificity for glucose in the same manner as Mueor-derived FAD-GDH described in Japanese Patent No. 4648993 as previously discovered by the inventors of the present invention.

Moreover, the mutants of the present invention were also determined to maintain high specific activity. An enzyme having high specific activity is preferable for use in applications to blood glucose sensors. The use of an enzyme having high specific activity makes it possible to complete measurement in a shorter amount of time by improving reactivity on the sensor. In addition, since advantages such as reductions in cost resulting from a reduction in the amount of enzyme used or reduced levels of measurement noise caused by contaminants are also expected, the development of an enzyme having high specific activity is extremely useful industrially.

1. A flavin-binding glucose dehydrogenase comprised of the amino acid sequence represented by SEQ ID NO: 1, an amino acid sequence having a sequence identity of 70% or more with the amino acid sequence represented by SEQ ID NO: 1, or an amino acid sequence in which one or a plurality of amino acids have been deleted, substituted or added in the amino acid sequence represented by SEQ ID NO: 1 or the amino acid sequence having a sequence identity of 70% or more with the amino acid sequence represented by SEQ ID NO: 1, having one or more amino acid substitutions at positions corresponding to amino acids selected from the group indicated below, and having improved heat stability in comparison with prior to carrying out that substitution:
the amino acid at position 66 in the amino acid sequence set forth in SEQ ID NO: 1,
the amino acid at position 68 in the amino acid sequence set forth in SEQ ID NO: 1,
the amino acid at position 88 in the amino acid sequence set forth in SEQ ID NO: 1,
the amino acid at position 158 in the amino acid sequence set forth in SEQ ID NO: 1,
the amino acid at position 233 in the amino acid sequence set forth in SEQ ID NO: 1,
the amino acid at position 385 in the amino acid sequence set forth in SEQ ID NO: 1,
the amino acid at position 391 in the amino acid sequence set forth in SEQ ID NO: 1, and
the amino acid at position 557 in the amino acid sequence set forth in SEQ ID NO: 1.

2. A flavin-binding glucose dehydrogenase comprised of the amino acid sequence represented by SEQ ID NO: 1, an amino acid sequence having a sequence identity of 70% or more with the amino acid sequence represented by SEQ ID NO: 1, or an amino acid sequence in which one or a plurality of amino acids have been deleted, substituted or added in the amino acid sequence represented by SEQ ID NO: 1 or the amino acid sequence having a sequence identity of 70% or more with the amino acid sequence represented by SEQ ID NO: 1, and having one or more of the amino acid substitutions at positions corresponding to amino acids selected from the group indicated below:
the amino acid at position corresponding to position 66 in the amino acid sequence set forth in SEQ ID NO: 1 is tyrosine,
the amino acid at position corresponding to position 68 in the amino acid sequence set forth in SEQ ID NO: 1 is glycine,
the amino acid at position corresponding to position 88 in the amino acid sequence set forth in SEQ ID NO: 1 is alanine,
the amino acid at position corresponding to position 158 in the amino acid sequence set forth in SEQ ID NO: 1 is histidine,
the amino acid at position corresponding to position 233 in the amino acid sequence set forth in SEQ ID NO: 1 is arginine,
the amino acid at position corresponding to position 385 in the amino acid sequence set forth in SEQ ID NO: 1 is threonine,
the amino acid at position corresponding to position 391 in the amino acid sequence set forth in SEQ ID NO: 1 is isoleucine, and
the amino acid at position corresponding to position 557 in the amino acid sequence set forth in SEQ ID NO: 1 is valine.

3. A flavin-binding glucose dehydrogenase in the form of a modified protein wherein the amino acid at the position corresponding to the asparagine residue at position 66 in an amino acid sequence composing a protein having flavin-binding glucose dehydrogenase activity indicated below is substituted with tyrosine:
a protein having flavin-binding glucose dehydrogenase activity comprised of the amino acid sequence set forth in SEQ ID NO: 1, or
a protein having flavin-binding glucose dehydrogenase activity comprised of amino acids in which one or a plurality of amino acids other than the amino acid residue at the position corresponding to the asparagine residue at position 66 in the amino acid sequence of SEQ ID NO: 1 has been deleted, substituted or added.

4. A flavin-binding glucose dehydrogenase in the form of a modified protein wherein the amino acid at the position corresponding to the asparagine residue at position 68 in an amino acid sequence composing a parent protein having flavin-binding glucose dehydrogenase activity indicated below is substituted with glycine:
a protein having flavin-binding glucose dehydrogenase activity comprised of the amino acid sequence set forth in SEQ ID NO: 1, or
a protein having flavin-binding glucose dehydrogenase activity comprised of amino acids in which one or a plurality of amino acids other than the amino acid residue at the position corresponding to the asparagine residue at position 68 in the amino acid sequence of SEQ ID NO: 1 has been deleted, substituted or added.

5. A flavin-binding glucose dehydrogenase in the form of a modified protein wherein the amino acid at the position corresponding to the cysteine residue at position 88 in an amino acid sequence composing a parent protein having flavin-binding glucose dehydrogenase activity indicated below is substituted with alanine:
a protein having flavin-binding glucose dehydrogenase activity comprised of the amino acid sequence set forth in SEQ ID NO: 1, or
a protein having flavin-binding glucose dehydrogenase activity comprised of amino acids in which one or a plurality of amino acids other than the amino acid residue at the position corresponding to the cysteine residue at position 88 in the amino acid sequence of SEQ ID NO: 1 has been deleted, substituted or added.

6. A flavin-binding glucose dehydrogenase in the form of a modified protein wherein the amino acid at the position corresponding to the threonine residue at position 158 in an amino acid sequence composing a parent protein having flavin-binding glucose dehydrogenase activity indicated below is substituted with histidine:
a protein having flavin-binding glucose dehydrogenase activity comprised of the amino acid sequence set forth in SEQ ID NO: 1, or
a protein having flavin-binding glucose dehydrogenase activity comprised of amino acids in which one or a plurality of amino acids other than the amino acid residue at the position corresponding to the threonine residue at position 158 in the amino acid sequence of SEQ ID NO: 1 has been deleted, substituted or added.

7. A flavin-binding glucose dehydrogenase in the form of a modified protein wherein the amino acid at the position corresponding to the glutamine residue at position 233 in an amino acid sequence composing a protein having flavin-binding glucose dehydrogenase activity indicated below is substituted with arginine:
a protein having flavin-binding glucose dehydrogenase activity comprised of the amino acid sequence set forth in SEQ ID NO: 1, or
a protein having flavin-binding glucose dehydrogenase activity comprised of amino acids in which one or a plurality of amino acids other than the amino acid residue at the position corresponding to the glutamine residue at position 233 in the amino acid sequence of SEQ ID NO: 1 has been deleted, substituted or added.

8. A flavin-binding glucose dehydrogenase in the form of a modified protein wherein the amino acid at the position corresponding to the alanine residue at position 385 in an amino acid sequence composing a protein having flavin-binding glucose dehydrogenase activity indicated below is substituted with threonine:
a protein having flavin-binding glucose dehydrogenase activity comprised of the amino acid sequence set forth in SEQ ID NO: 1, or
a protein having flavin-binding glucose dehydrogenase activity comprised of amino acids in which one or a plurality of amino acids other than the amino acid residue at the position corresponding to the alanine residue at position 385 in the amino acid sequence of SEQ ID NO: 1 has been deleted, substituted or added.

9. A flavin-binding glucose dehydrogenase in the form of a modified protein wherein the amino acid at the position corresponding to the leucine residue at position 391 in an amino acid sequence composing a protein having flavin-binding glucose dehydrogenase activity indicated below is substituted with isoleucine:
a protein having flavin-binding glucose dehydrogenase activity comprised of the amino acid sequence set forth in SEQ ID NO: 1, or
a protein having flavin-binding glucose dehydrogenase activity comprised of amino acids in which one or a plurality of amino acids other than the amino acid residue at the position corresponding to the leucine residue at position 391 in the amino acid sequence of SEQ ID NO: 1 has been deleted, substituted or added.

10. A flavin-binding glucose dehydrogenase in the form of a modified protein wherein the amino acid at the position corresponding to the leucine residue at position 557 in an amino acid sequence composing a protein having flavin-binding glucose dehydrogenase activity indicated below is substituted with valine:
a protein having flavin-binding glucose dehydrogenase activity comprised of the amino acid sequence set forth in SEQ ID NO: 1, or
a protein having flavin-binding glucose dehydrogenase activity comprised of amino acids in which one or a plurality of amino acids other than the amino acid residue at the position corresponding to the leucine residue at position 557 in the amino acid sequence of SEQ ID NO: 1 has been deleted, substituted or added.

11. A flavin-binding glucose dehydrogenase wherein the amino acids at positions corresponding to the amino acid sequence represented by SEQ ID NO: 1, an amino acid sequence having a sequence identity of 70% or more with the amino acid sequence represented by SEQ ID NO: 1, or an amino acid sequence in which one or a plurality of amino acids in the amino acid sequence represented by SEQ ID NO: 1 or the amino acid sequence having a sequence identity of 70% or more with the amino acid sequence represented by SEQ ID NO: 1 have been deleted, substituted or added, are any of the amino acid residues set forth below:
the amino acid at the position corresponding to asparagine at position 66 in the amino acid sequence set forth in SEQ ID NO: 1 is tyrosine, and the amino acid at the position corresponding to asparagine at position 68 is glycine,
the amino acid at the position corresponding to cysteine at position 88 in the amino acid sequence set forth in SEQ ID NO: 1 is alanine, the amino acid at the position corresponding to asparagine at position 66 is tyrosine, and the amino acid at the position corresponding to asparagine at position 68 is glycine,
the amino acid at the position corresponding to cysteine at position 88 in the amino acid sequence set forth in SEQ ID NO: 1 is alanine, and the amino acid at the position corresponding to threonine at position 158 is histidine,
the amino acid at the position corresponding to cysteine at position 88 in the amino acid sequence set forth in SEQ ID NO: 1 is alanine, and the amino acid at the position corresponding to glutamine at position 233 is arginine, or
the amino acid at the position corresponding to cysteine at position 88 in the amino acid sequence set forth in SEQ ID NO: 1 is alanine, the amino acid at the position corresponding to leucine at position 557 is valine, and the amino acid at the position corresponding to serine at position 559 is lysine.

12. The flavin-binding glucose dehydrogenase according to any of claims 1 to 11, provided with the properties described in (I) and/or (II) below:
(I) having a residual activity rate of 50% or more following heat treatment for 15 minutes at pH 7.0 and 40° C., and/or
(II) having a ratio of reactivity with D-xylose to reactivity with D-glucose (Xyl/Glc(%)) of 2% or less.

13. A flavin-binding glucose dehydrogenase gene encoding the flavin-binding glucose dehydrogenase according to claim 1.

14. A recombinant DNA comprising the flavin-binding glucose dehydrogenase gene according to claim 13 inserted into vector DNA.

15. A host cell introduced with the recombinant DNA according to claim 14.

16. A method for producing flavin-binding glucose dehydrogenase, comprising the following steps:
a step for culturing the host cell according to claim 15,
a step for expressing a flavin-binding glucose dehydrogenase gene contained in the host cell, and
a step for isolating the flavin-binding glucose dehydrogenase from the culture.

17. A glucose measurement method using the flavin-binding glucose dehydrogenase according to claim 1.

18. A glucose assay kit containing the flavin-binding glucose dehydrogenase according to claim 1.

19. A glucose sensor containing the flavin-binding glucose dehydrogenase according to claim 1.


SEQUENCE LISTING (20 sequences)

SEQ ID NO: 1 - protein, 641 aa, Mucor prainii [sequence omitted]

SEQ ID NO: 2 - DNA, 1926 bp, Mucor prainii
atgaagatcacagctgccattatcactgttgccacagcatttgcttcttttgcttctgctcaacaagacacaaattcttcctcaactgatacttatgattatgttatcgttggcggcggtgtagctggtttggctttggctagtcgtatctctgaaaacaaggatgtcactgttgctgttctcgagtccggtcctaatgccaatgatagatttgttgtttatgctcctggcatgtatggccaagctgttggcactgatctctgtcctctcattcctactactcctcaagaaaatatgggcaacagaagtctcacaatcgctactggtagattgctcggtggtggcagtgctattaatggtctcgtttggacccgtggtggcttgaaggattacgatgcttgggaggagctcggtaaccctggatggaacggtgccaacttgttcaagtactttaagaaggtcgaaaacttcactcctcctactcctgcccaaattgaatacggcgctacttatcagaaaagtgctcatggcaagaagggacctattgatgtctctttcacgaactacgagttctctcaatctgctagctggaacgcctcactcgaaacccttgatttcactgcacttcctgatatcttgaacggtactttggccggttactctaccactcccaacattttggaccctgagactgttcaacgtgttgattcctatactggttacattgctccttacactagccgtaacaacctcaatgttttggccaaccataccgtctcccgcattcaatttgctcccaagaatggtagcgaacctctcaaggctaccggtgttgaatggtatcccactggcaacaagaatcaaaagcaaattatcaaggcccgttatgaagttatcatctcatctggtgccattggtagtcctaagcttttggaaatctctggtatcggtaataaggatatcgtctctgctgctggtgtcgagtccttgattgacttgcctggcgttggttccaacatgcaagatcacgttcatgctatcactgtctctactaccaatattactggctatactaccaacagcgtctttgtcaatgaaacccttgcccaagaacaaagagaagaatatgaagccaacaagactggtatctgggctactactcccaacaacctcggttatcctacgcccgaacaactcttcaatggcaccgaattcgtttctggaaaggagtttgctgacaagattcgtaactctactgatgaatgggccaactattatgcttccaccaacgcctccaatgtcgagttattaaagaagcaatatgctattgtcgcctctcgttacgaagagaactacttgtctcctattgaaatcaacttcactcctggttatgagggtagcggtaatgtcgatttgcaaaacaacaagtaccaaactgtcaaccatgtcttgattgctcctttaagtcgtggttatactcacattaactcttctgatgtggaggatcattctgtcattaatccccaatactactctcatcctatggatattgatgtccatatcgcttccactaaacttgctcgcgaaatcatcactgcctctcccggtcttggtgacattaacagtggcgaaatcgaacccggtatgaatattacttctgaagacgaccttagatcttggttgagtaataatgtccgttctgactggcatcctgttggtacttgtgctatgcttcccaaggaattaggtggtgttgtcagccccgctctcatggtttacggcacttccaacttgcgtgttgttgatgcttcgattatgcccctcgaagtctcttctcatttgatgcaacccacctacggtattgctgagaaggctgctgacattattaagaatttctacaagactcaacacaagaaccaaaattag

SEQ ID NO: 3 - DNA, 30 bp, Artificial Sequence
  Artificially Synthesized Primer Sequence
ggtccttatgccggtgatagatttgttgtt

SEQ ID NO: 4 - DNA, 30 bp, Artificial Sequence
  Artificially Synthesized Primer Sequence
catgccaggagcataaacaacaaatctatc

SEQ ID NO: 5 - DNA, 30 bp, Artificial Sequence
  Artificially Synthesized Primer Sequence
ggcactgatctcgctcctctcattcctact

SEQ ID NO: 6 - DNA, 30 bp, Artificial Sequence
  Artificially Synthesized Primer Sequence
attttcttgaggagtagtaggaatgagagg

SEQ ID NO: 7 - DNA, 30 bp, Artificial Sequence
  Artificially Synthesized Primer Sequence
gtcgaaaacttccatcctcctactcctgcc

SEQ ID NO: 8 - DNA, 30 bp, Artificial Sequence
  Artificially Synthesized Primer Sequence
gccgtattcaatttgggcaggagtaggagg

SEQ ID NO: 9 - DNA, 30 bp, Artificial Sequence
  Artificially Synthesized Primer Sequence
cctgagactgttcgacgtgttgattcctat

SEQ ID NO: 10 - DNA, 30 bp, Artificial Sequence
  Artificially Synthesized Primer Sequence
agcaatgtaaccagtataggaatcaacacg

SEQ ID NO: 11 - DNA, 30 bp, Artificial Sequence
  Artificially Synthesized Primer Sequence
gacgacgttagaaaatggttgagtaataat

SEQ ID NO: 12 - DNA, 27 bp, Artificial Sequence
  Artificially Synthesized Primer Sequence
gtcagaacggacattattactcaacca

SEQ ID NO: 13 - DNA, 29 bp, Artificial Sequence
  Artificially Synthesized Primer Sequence
ggtccttatgccaatgatagatttgttgt

SEQ ID NO: 14 - DNA, 30 bp, Artificial Sequence
  Artificially Synthesized Primer Sequence
ggtcctaatgccggtgatagatttgttgtt

SEQ ID NO: 15 - DNA, 30 bp, Artificial Sequence
  Artificially Synthesized Primer Sequence
actcccaacaacatcggttatcctacgccc

SEQ ID NO: 16 - DNA, 30 bp, Artificial Sequence
  Artificially Synthesized Primer Sequence
attgaagagttgttcgggcgtaggataacc

SEQ ID NO: 17 - DNA, 30 bp, Artificial Sequence
  Artificially Synthesized Primer Sequence
gacgacgttagatcttggttgagtaataat

SEQ ID NO: 18 - DNA, 30 bp, Artificial Sequence
  Artificially Synthesized Primer Sequence
gacgaccttagaaaatggttgagtaataat

SEQ ID NO: 19 - DNA, 30 bp, Artificial Sequence
  Artificially Synthesized Primer Sequence
actggtatctggactactactcccaacaac

SEQ ID NO: 20 - DNA, 30 bp, Artificial Sequence
  Artificially Synthesized Primer Sequence
cgtaggataaccgaggttgttgggagtagt